(19) United States
(12) Patent Application Publication
(10) Pub. No.: US 2016/0331279 A1
(43) Pub. Date: Nov. 17, 2016

(54) METHODS FOR DETERMINING PSYCHOLOGICAL CHARACTERISTICS AND GENDER USING MOTION-BASED ANALYSIS, AND RELATED METHODS FOR TARGETING ADVERTISING AND IMPROVING RELEVANCE OF USER REVIEWS

(71) Applicants: Pavel Shpin, Cupertino, CA (US); Aleksander Tetkin, Novokuznetsk (RU); Anton Bondar, Cupertino, CA (US)

(72) Inventors: Pavel Shpin, Cupertino, CA (US); Aleksander Tetkin, Novokuznetsk (RU); Anton Bondar, Cupertino, CA (US)

(73) Assignee: Nexinity Inc, Cupertino, CA (US)

(21) Appl. No.: 14/711,724

(22) Filed: May 13, 2015

(51) Int. Cl. A61B 5/11

(52) U.S. Cl. CPC A61B 5/11; A61B 5/1118; A61B 5/112

(57) ABSTRACT

Methods for determining psychological characteristics and gender using motion-based analysis, and related methods for targeting advertising and improving relevance of user reviews.

### CROSS REFERENCE TO RELATED APPLICATION

This application claims priority from U.S. Provisional Patent Application No. 61/992,660 filed on May 13, 2014 entitled METHODS FOR DETERMINING PSYCHOLOGICAL CHARACTERISTICS AND GENDER USING MOTION-BASED ANALYSIS, AND RELATED METHODS FOR TARGETING ADVERTISING AND IMPROVING RELEVANCE OF USER REVIEWS, which is hereby incorporated by reference.

### FIELD OF THE INVENTION

The inventive solutions disclosed herein relate to the field of applied psychology. More particularly, disclosed are methods for determining psychological characteristics and gender of individuals, and utilizing individual's psychological characteristics and gender data for advertising targeting, as well as improving the relevance of user reviews of products and services.

### Description of the Related Art

Modern psychology describes the psychological characteristics of people, such as perception, introspection, memory, creativity, imagination, idea, belief, reasoning, volition, and emotion, using a unified system of predefined psychological characteristics. For example, renowned psychologist Carl Gustav Jung introduced a concept of psychological types, later developed by his many followers. The most well-established psychological characteristics are dichotomy sensing-intuition, thinking (logic)-feeling (ethics), extroversion-introversion, rationality-irrationality, however, different theories may have different meanings for psychological characteristics of the same name (e.g. between the theories Carl Gustav Jung and Hans Jürgen Eysenck). Various systems of psychological characteristics exist.

For the purpose of clarification, as used herein, a set of specified values of certain psychological characteristics forms a “psychological profile” of an individual. It is believed that people with the same psychological profile tend to absorb, process; evaluate and judge information and draw conclusions in a similar manner, as well as generally possess a similar model of social behavior. Psychological profiles, according to different theories, are also referred to as <<Personality types>>—in Mayers-Brigs Type Indicator (MBTI); or <<Sociotypes>>, <<Types of information metabolism>>—in Socionics.

The Myers-Briggs Type Indicator (MBTI) assessment is a psychometric questionnaire designed to measure psychological preferences in how people perceive the world and make decisions. These preferences were extrapolated from the typological theories first proposed by Carl Gustav Jung, who suggested that there are four principal psychological functions by which we experience the world: sensation, intuition, feeling, and thinking. One of these four functions is dominant most of the time.

Socionics, in psychology and sociology, is a theory of information processing and personality type, distinguished by its information model of the psyche and a model of interpersonal relations. Socionics is a modification of Jung's personality type theory that uses eight psychic functions, in contrast to Jung's model, which used only four. These functions process information at varying levels of competency and interact with the corresponding function in other individuals, giving rise to predictable reactions and impressions—a theory of intertype relations

It often beneficial to know the information of an individual's psychological profile. This information is often used for the purposes of professional orientation and assessment, creating motivational schemes and providing psychological assistance as well as many other uses.

The information about the psychological profile and gender can also be effectively used for the positioning of products and services, targeting of individual advertising in view of the objectively anticipated affinity or preferences to an particular product, service or advertising message itself.

As used herein “individual advertising” refers to advertising being addressed to particular person (even if anonymously), including online advertising, advertising on the screens of personal devices, advertising on the screens of devices with user authorization, and other types of personally-addressed media.

Conventional methods for determining of psychological characteristics (psychological profile) of individuals employ two main approaches:
Expert-based determination—assessment by a trained person, e.g. psychologist
Test-based—assessment using various tests and questionnaires by analyzing the answers and drawing conclusions.

In both types of methods, determining the psychological characteristics of the individual requires the involvement of experts and/or individuals being tested. This requirement presents an obstacle to the large-scale determination of psychological profiles of people.

U.S. Pat. No. 6,006,188 A issued to Alexeev describes the method of speech signal processing for determining psychological or physiological characteristics using a knowledge base. Alexeev provides a speech-based system for assessing psychological, physiological or other characteristics of a test subject. The system includes a knowledge base that stores one or more speech models, where each speech model corresponds to a characteristic of a group of reference subjects. The limitation of this method is using the speech of individuals which is not always possible due to privacy concerns or requires attention of test subject.

Advertising targeting is the set of methods and techniques used to improve the effectiveness of advertising campaigns by narrowing the audience, which is delivered (demonstrates) advertising. Knowing of the psychological profile of advertisement recipients and their gender can improve the quality of advertising targeting; however described approaches to the definition of psychological profiles do not support a broad application of information about psychological profiles of individuals for these purposes.

U.S. Pat. Appl. Publication No. US20130275230 A1 by Sawyer describes methods and systems for targeted advertising based on passively collected sensor-detected offline behavior. Though used wide variety of sensor in this approach, the limitation of it is to identify particular behavior pattern in given context and to identify the particular needs of the persons for advertisement targeting. When in method provided identified the psychological profile which is more broad and allows to use the way of thinking, perception, judging and decision making of individuals.

### SUMMARY OF THE INVENTION

Inventive solutions disclosed herein employ determination of psychological parameters, characteristics and gender of individuals based on an analysis of her or her motions over time. Thusly determined psychological profiles can then be used in a number of commercial applications, such as, for example, targeted advertising and improving relevance of users reviews of products and services for presentation to other users.

The determining the values of psychological characteristics and gender of individuals are based on an analysis of their motions and other nonverbal determining indicators or cues of these characteristics. As contemplated by the inventors herein, data about individual's motions passively detected by motion sensors built into wearable devices, such as smartphones, or dedicated electronic, or electromechanical special-purpose units. The collected sensor data analyzed in order to identify the pattern related to people with certain psychological characteristics or gender. Related patterns of certain psychological characteristics or gender identified by analytical methods and/or methods of machine learning on a group of people which psychological characteristics are then determined by traditional methods—by the experts or test-based.

The method for determining psychological profiles disclosed herein can be used to identify both “personality types” according to MBTI and “sociotypes” according to socionics theory. Both of these psychological classifications referenced above based on four widely used psychological characteristics:

Extraversion—introversion

Sensing—intuition

Thinking(logic)—feeling (ethics)

Irrational—rational

Each of psychological characteristics listed above reflected in non-verbal behavior of individuals (motions).

For people classified as “sensing” (in opposite to “intuition”):
Gait is firmer
Less fussy
Motions slower
When resting the fluctuations of body is less
Decay of the vibrational motions (fluctuations) faster
Frequency of the motions is less
Disperse of body positions is less
   For people classified as “rational” (in opposite to “irrational”)
Disperse of main frequencies of cyclic motions is less
The disperse of energy of motions along different axis is less
   For people classified as “extraverted” (in opposite to “introverted”)
The average speed of motions is higher
The average quantity (amount?) of motions is more
   For people classified as “logic” (in opposite to “ethics”)
The accelerations of motions is higher
Motions more discrete

Typically, the motions of the head gives more precise information than other parts of the body.

Parallel gathering data from multiple sensors placed in different parts of the body also increase precise of determination (e.g. smartphone in the pocket, fitness tracker on the wrist and smart glasses on the head).

Since the signs of characteristics are very vague, the machine learning algorithms is the best to detect and classify.

Methods of using psychological characteristics (psychological profile) and gender for advertisement targeting based on the narrowing of the audience to display advertisement based on matching the psychological and gender profile of advertising and psychological and gender profile of the viewer. Psychological profile of the viewer is determined according to the method. Psychological profile of an advertisement—a set of values of psychological characteristics of users, who most positively responded to this advertisement. Gender profile of an advertisement—a gender information of users, who most positively responded to this advertisement. The first method—the psychological profile and gender profile of future advertisement is predefined. Advertisement should be created to specific perceptions in accordance of holders of certain psychological characteristics or gender using the marketing and designing techniques to increase their interest in the advertised product or service. The second method is statistical and does not require special preparation of advertisement. Advertisement shows to a relatively small number of users (test group) with known psychological profiles and(or) gender which tracked their reactions. Analyzing the reactions of representatives in the test group revealed a statistical relationship. Holders of psychological characteristics or gender that are most responsive to advertising (click on the banner ad, pass on the proposed internet link or other traceable method of positive reaction to advertising) determine the orientation of the corresponding advertising to certain psychological profiles. Once the psychological profile and gender profile of an advertisement is determined, the advertisement should be shown to users with this or similar psychological profile. The continued gathering of statistics allows adjustment to the psychological profile or gender profile for the advertisement.

Method of choosing reviews of products and services for presentation to the user based on the fact that the owners of similar psychological characteristics share similar ways to absorb information, process and draw conclusions, have a similar model of social behavior. It also means that people with similar psychological characteristics are similar evaluating the quality and characteristics of products and services, and their opinions about products and services, their approach to expressing opinions and understanding are also similar. The possibility to demonstrate product reviews and services on websites, mobile and desktop applications within groups of people with similar psychological characteristics will create more value for users comments and provide more relevance to the reviews, which increases the attractiveness for the user and website visitor.

### BRIEF DESCRIPTION OF THE DRAWINGS

FIG. 1 is a block diagram illustrating the process of detecting motion patterns related to particular psychological characteristic or gender.

FIG. 2 is a block diagram illustrating one of the embodiments of the invention—advertising targeting process applied to wearable device with built-in display such as smartphone.

FIG. 3 is a block diagram illustrating one of the embodiments of the invention—advertising targeting process applied to paired devices, one of them used for gathering motion sensors data and other used for identifying psychological profile and to display advertisement.

FIG. 4 is a block diagram illustrating the statistical method advertising targeting.

FIG. 5 is a block diagram illustrating one of embodiments of invention in advertising targeting for Web.

### DETAILED DESCRIPTION OF THE INVENTION

Determining of Psychological Characteristics and Gender

As mentioned above, the inventive solution contemplated by the inventors is based on determining the values of psychological characteristics (i.e. a psychological profile) and gender based on the data obtained via motion sensors, such as, for example, accelerometers and gyroscopes embedded in wearable devices, including, but not limited to—phones, smart phones, smart watches, fitness trackers, medical device monitoring of the human condition, smart glasses, etc. Thusly obtained data may then be processed via analytical methods, via machine learning methods, or both. Other types motion sensors are contemplated by the invention.

The data from a sensor can be acquired in frequency range 2-50 Hz depending on psychological characteristic determined.

The analytical approach used in cases when it is known how specific psychological characteristics and gender are expressed in human motions. Data from motion sensors processed by the computer program, which implement the algorithm determining values of these characteristics in accordance with knowledge of how certain psychological characteristics and gender are expressed in human motions.

The approach of using machine learning is applied when the analytical approach cannot or should not be implemented because of inaccuracy or inefficiency, e.g. when it is not possible to precisely describe how certain psychological characteristics and gender are expressed in human motions. Experienced psychologists are able to use non-verbal signs to determine these characteristics, but describing as a set of precise rules how accomplish it is not always possible due to the complex fuzzy pattern. Supervised machine learning algorithms can be used in this case. Since the most of the simple psychological characteristics have a limited set of possible values (e.g. true or false, gender—male or female), it becomes a well-known classification problem. To find a solution for this classification problem a large variety of machine learning algorithms can be used—artificial neural networks, support vector machine, radial basis network, the wavelet neural networks, etc. Training data consists of a set of training examples. Each example is a pair consisting of input data and known value of psychological characteristics or gender. The known values of psychological characteristics and gender can be obtained by traditional methods to determine peoples psychological characteristics, as input data uses data received from motion sensors produced depending on the machine learning algorithm.

Data to feed and train the machine learning algorithms must be prepared taking in consideration that it is a signal, respectively, may be using methods of statistical signal processing, Fourier transform, wavelet transform, and other signal feature extraction techniques. Some types of machine learning algorithms require data to be normalized and others, e.g. a wavelet neural networks may accept data in its original form. In order to reduce the overfitting effect in some cases it is necessary to pre-filter the signal, by excluding low-information parts of the frequency spectrum. Excluded from the analysis sensor data relating to an individual activity in which, from the point of view of experts, analyzed the characteristic is not expressed or weakly expressed, e.g. driving an automobile.

To determine the values of psychological characteristics and gender as input of machine learning algorithms can be used with the following parameters of the motion sensor signals (but not limited to this list):

The rate of decay of the vibrational motions (linear, angular, by each axis, by each combination of axis)
Dispersion of positions
Range of motions (linear, angular, by each axis, by each combination of axis)
The frequencies of motions (linear, angular, by each axis, by each combination of axis)
Statistical assessment of amount of motions for every degree of freedom
Linear and angular speeds (by each axis, by each combination of axis)
Linear and angular accelerations (by each axis, by each combination of axis)
Statistical assessment of values above—mean, mode, disperse, median, skewness, kurtosis, central moment, etc.

Some psychological characteristics can be determined on the analysis of motions for a relatively long period of time measured in hours or days. This is especially true of psychological characteristics that can be identified in the analysis of motions in individual's scheduled activities, such as regular walking. The analysis may be a study of the degree of difference in gait over time.

Some psychological characteristics and gender can be better expressed and more qualitatively determined from the data with motion sensors, obtained by certain human activities, such as analysis of the head motions during a phone call conversation, gait analysis during rectilinear walking.

Most of characteristics listed above are changing in time, and for the purpose of recognition statistic-temporal pattern as the feed of machine learning algorithms the series of values in selected time frame should be provided. Time frame depends on particular characteristic identified.

Mixed methods provided—the part of psychological characteristics can be determined by the analytical method, the other part—the method of machine learning.

The implementation may be software, hardware, firmware, as part of an integrated circuit or a programmable logic array.

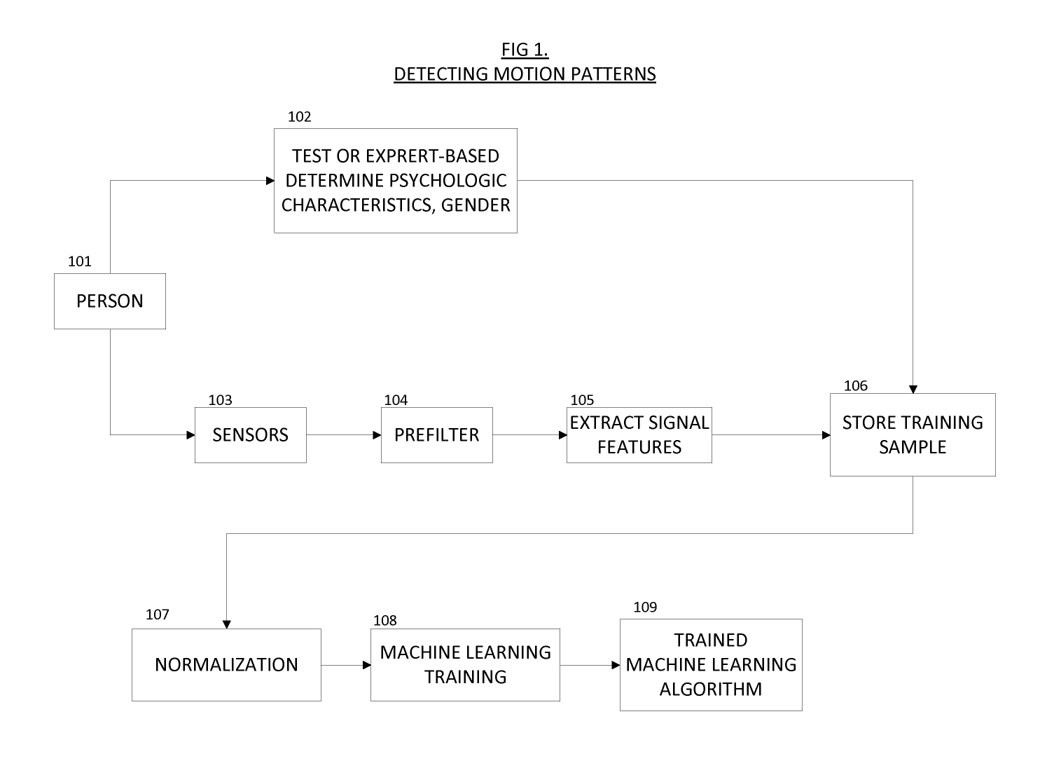

FIG. 1. is a block diagram illustrating one of embodiments of the process of detecting motion patterns related to particular psychological characteristic or gender. The psychological characteristics and gender of person 101 is preliminary determined at block 102 during test of expert-based procedure. Psychological characteristics and gender is stored in the database at 106. The motion sensors 103 detect motions of tested individual 101 and pass the data to the filter 104. Filter 104 removes noises and useless data from the signal data. Then at the block 105 prefiltered data is processed to extract signal features. Extracted features stored in the database 106 are paired with the data acquired at block 102.

When enough data is collected at block 106 to train certain machine learning algorithm, the collected data is normalized at block 107 if it is necessary for the chosen machine learning algorithm. After normalization, the data is passed to machine learning train stage 108 to detect motion patterns accorded to certain psychological characteristic or gender.

The result of training is the trained machine learning algorithm 109 which is the data used later to determine psychological characteristics or gender. This data can be stored in some database or hardcoded in software, hardware or firmware solutions.

Result of the determination can be stored in the memory of a personal wearable device or to be transferred to the server database of an advertising provider and stored there in association with a specific personal wearable device (unique device identifier), which will be used to display advertising. The information about psychological and gender profile may also be stored on a server database in association with one or more accounts registered to a smartphone for interaction with various social media or cloud storage services.

Motion sensors can be built into the devices that will be used to display advertisements.

Motion sensors may also be built into wearable devices associated with a device for displaying advertisement with a communication channel between these devices. For example, it may be a smartphone equipped with motion sensors, which are used for motion detection of the user, or smartphone can be connected (including wirelessly) to fitness tracker, which transmits that information about the motion to the smartphone.

Results of the determination of psychological characteristics can be stored locally on the device or transmitted to the server database of advertising provider for use in targeting. As the keys for storing information about the psychological characteristics of the database provider advertising can be used on a unique number of personal devices, user accounts on different sites and services.

### Advertisement Targeting

The inventive solutions disclosed herein further contemplate targeting of advertising content to particular viewers based on their psychological characteristics and gender, as determined using the methods described above.

In some embodiments; content of advertisement is initially created aimed at holders of certain psychological characteristics and/or gender by following expert recommendations and/or assistance. Alternatively, an already-prepared advertisement content can be analyzed to determine suitable psychological or gender profile of its audience. The group of viewers with psychological or gender-matching profiles is the target audience for this advertisement.

Information about advertisement and the target audience is stored in a database advertisement provider.

An advertisement provider can be any company in the chain of the advertisement delivery to the user.

The software which is used to display advertising on mobile devices (e.g. Mobile Advertisement SDK) transmits psychological characteristics to advertising providers, or transmit raw or preprocessed data signals from sensors, or transmits device identification number or user accounts, allowing the advertising provider to establish the connection between the device or user and psychological characteristics, therefore providing the advertisement matched with viewers psychological profile to increase the probability to be positively accepted by the user.

When displaying advertisements on web sites on the server side the advertisement to display should be selected by matching user and advertisement psychological profiles which are stored in the database. As the key for searching in the database can be used for user account information, which can be obtained during the user authorization process, sent to the server as part of browser cookies, transmitted by direct request to the server or with a specific unique identifier of holders of certain psychological profiles. This specific unique identifier may be generated during passing the regular psychological test or questionnaire of determining user's psychological profile and stored in browser storage environment such as browser cookies or browser embedded database.

Individual's psychological and gender profile identified by using motion sensors of wearable device which stored in database with key as account identifier on web services (if it allowed for the application gathering motion sensor data), can be used with the same advertisement targeting technique on web sites by requesting relevant advertisement from server using account information of this web service.

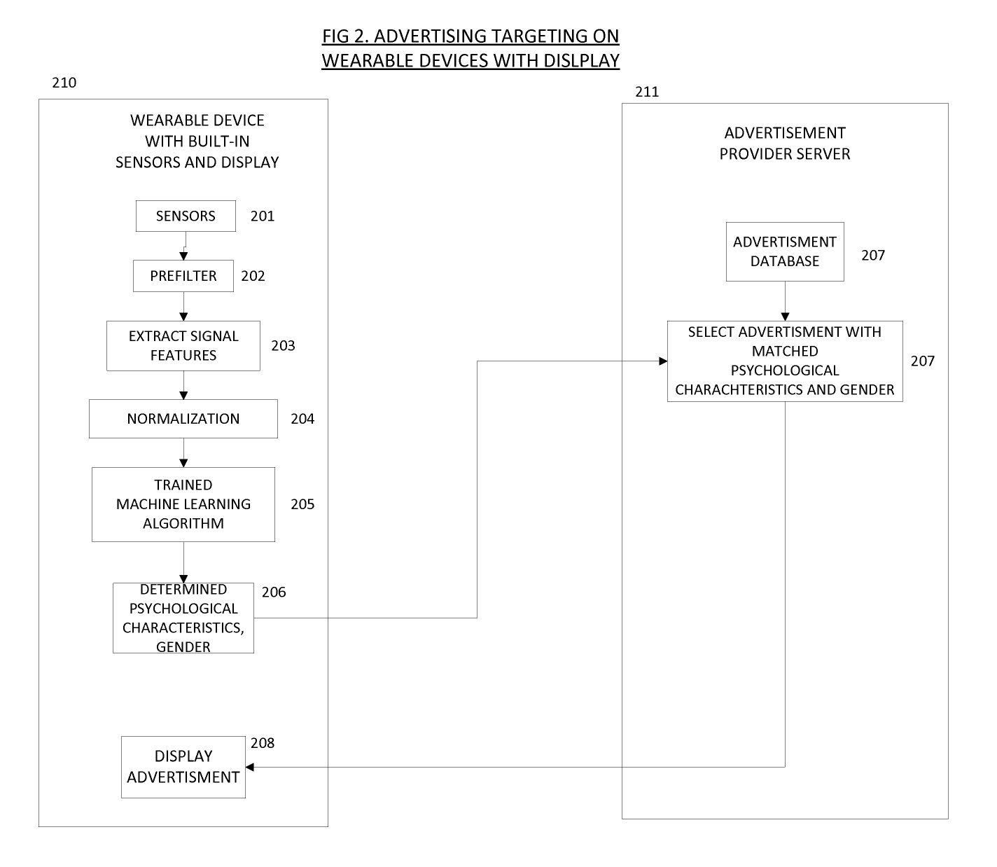

FIG. 2. Illustrates one of embodiments of invention in advertising targeting on wearable devices with display.

Wearable device 210 with built-in sensors and display is used by an individual. Motion sensors 201 register motion and pass data to filter 202. Filter 202 removes noises and useless data from the signal data. Then at the stage 203 prefiltered data is processed to extract signal features. The data, as results of the processing at the stage 203 should be processed in the same manner as it does at stage 105 of FIG. 1.

Data from 203 is normalized at 204 if it is necessary for the chosen machine learning algorithm.

After normalization the data is passed as input to trained machine learning algorithm 205 to detect psychological characteristics and/or gender of the individual.

Determined psychological characteristics and/or gender are passed to advertisement provider server 211 to select advertisement with matched psychological characteristics and gender of the advertisement stored in database 207.

Selected advertisement is passed to the wearable device 210 to display at 208.

In one or more embodiments, the stages 203, 204, 205, 206 can be implemented at server side 211.

In one or more embodiments, the stages 201-206 and 208 can be implemented in different applications on the same device or applications on different devices with passing data through the advertisement server using some identifier that can help establish association between different applications or devices for the same user, it can be device identifiers, user accounts, advertiser Identifier etc.

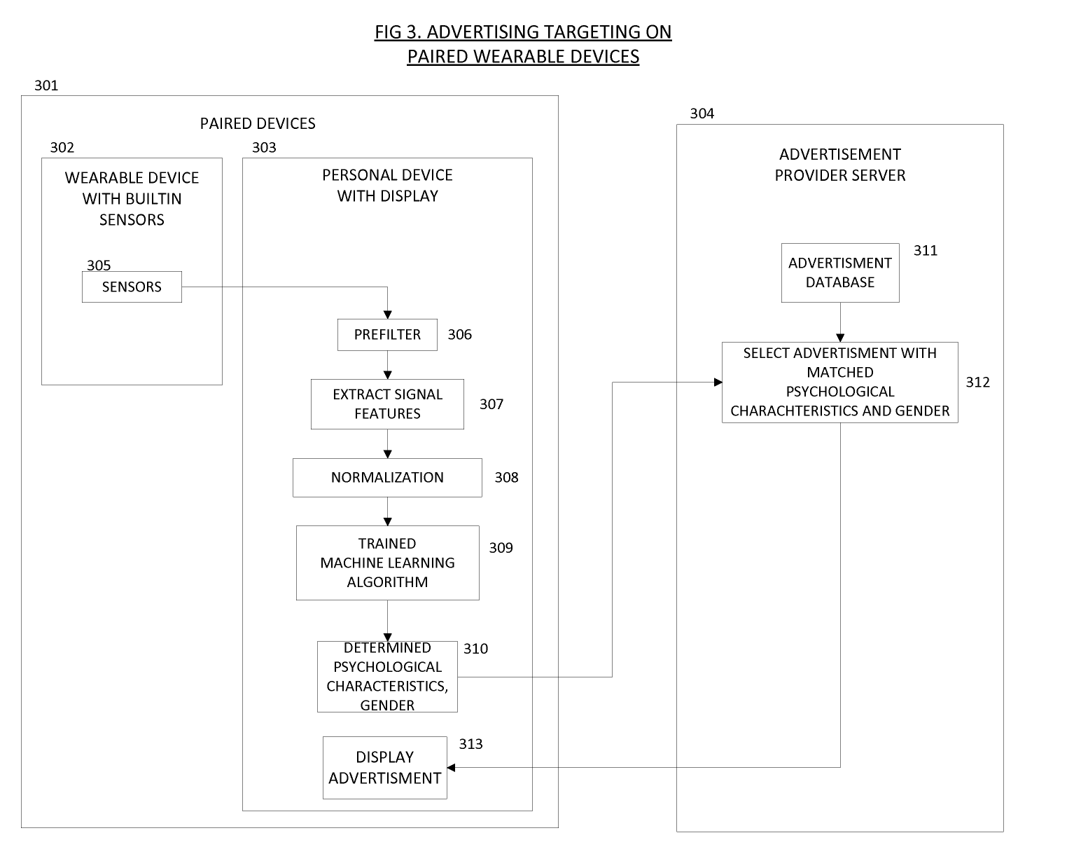

FIG. 3. Illustrates one of embodiments of invention in advertising targeting on paired wearable devices.

This embodiment is particularly suitable when one of the devices with motion sensors is used for detecting individual's motions, and other is used to display advertisement directed to the same person.

Wearable device 302 with built-in motion sensors is used by the individual. Device 302 could be the fitness tracker, smartwatch, glasses, headset or any other device with motion sensors capable to detect individual's motions.

Motion sensors 305 detect individual's motions. Device 302 passes sensors' data via communication channel to the other personal device 303 equipped with display and capabilities to communicate with advertisement provider server 304.

Motion sensors' data from sensors 305 is passed to personal device 303 to the prefiltering stage 306 to remove noises and useless data from the signal data. Then at the stage 307 prefiltered data is processed to extract signal features. The data as results of the processing at the stage 307 should be processed in the same manner as it does at stage 105 of FIG. 1.

Data from 307 is normalized at 308 if it is necessary for the chosen machine learning algorithm.

After normalization, the data is passed as input to trained machine learning algorithm 309 to detect psychological characteristics and/or gender of the individual.

Determined psychological characteristics and/or gender are passed to advertisement provider server 312 to select advertisement with matched psychological characteristics and gender of the advertisement stored in database 311.

Selected advertisement is passed to wearable device 303 to display at 313.

In one or more embodiments the stages 306, 307, 308, 309, 310 can be implemented inside wearable device 302 or at the server side 311.

### Statistical Method

Targeting is performed in two stages—identifying the target audience for ready advertisement, display advertising to the identified target audience.

To identify the target audience of the advertisement should be shown to small test group of viewers, in which the amount of holders of each psychological and gender profile are similar, tracking the reaction of the representatives of each psychological and gender profile on the advertisement. Those psychological and gender profiles, whose representatives were the most positive in response to this advertisement, become identified be as the target audience for the rest of advertising campaign. Information about the target audience for advertisement is stored in a database and used later to eliminate the first stage in the next campaign with the same advertisement. Since target audience identified the rest of advertisement campaign applied mostly or only to the target audience.

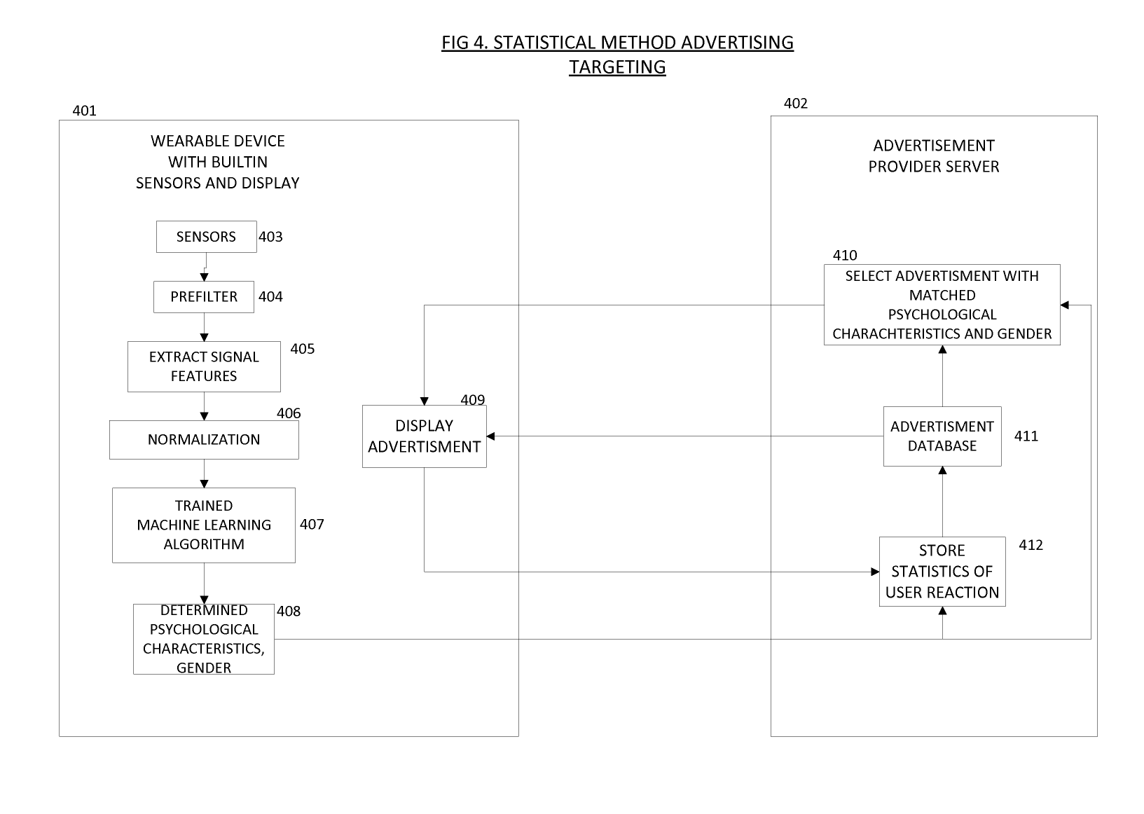

FIG. 4. Illustrates one of embodiments of the statistical method described in invention in advertising targeting on wearable devices with display.

Wearable device 401 with built-in sensors and display is used by an individual. Motion sensors 403 register motion, then data is passed to filter 404. Filter 404 removes noises and useless data from the signal data. Then at the stage 405 prefiltered data is processed to extract signal features. The data as results of the processing at the stage 405 should be processed in the same manner as it does at stage 105 of FIG. 1.

Data from stage 405 is normalized at stage 406 if it is necessary for the chosen machine learning algorithm.

After normalization the data is passed as input to trained machine learning algorithm 407 to detect psychological characteristics and/or gender of the individual.

Determined psychological characteristics and/or gender are passed to advertisement provider server 402 to store in 412.

When psychological characteristics and/or gender are stored at 412 the stage of identifying the target audience by gathering statistics fires. In this stage advertisement provider server 402 sends a random advertisement from database 411 to wearable device 401 to display it at 409. The user's reaction is sent to server 402 to store in 412. After displaying the preset a certain amount of times (some fraction of the total advertisement campaign advertisement showing) the stage of identifying of the target audience is completed. The target audience data stores in the advertisement database 411.

Since target audience for certain advertisement is stored in database 411, the rest of the advertisement campaign is performed mostly or only to the target audience by selecting the advertisement from database 411 at 410 with specified psychological characteristics or gender data received from 408.

In one or more embodiments the stages 408, 406, 407, 408 can be implemented at server side 402.

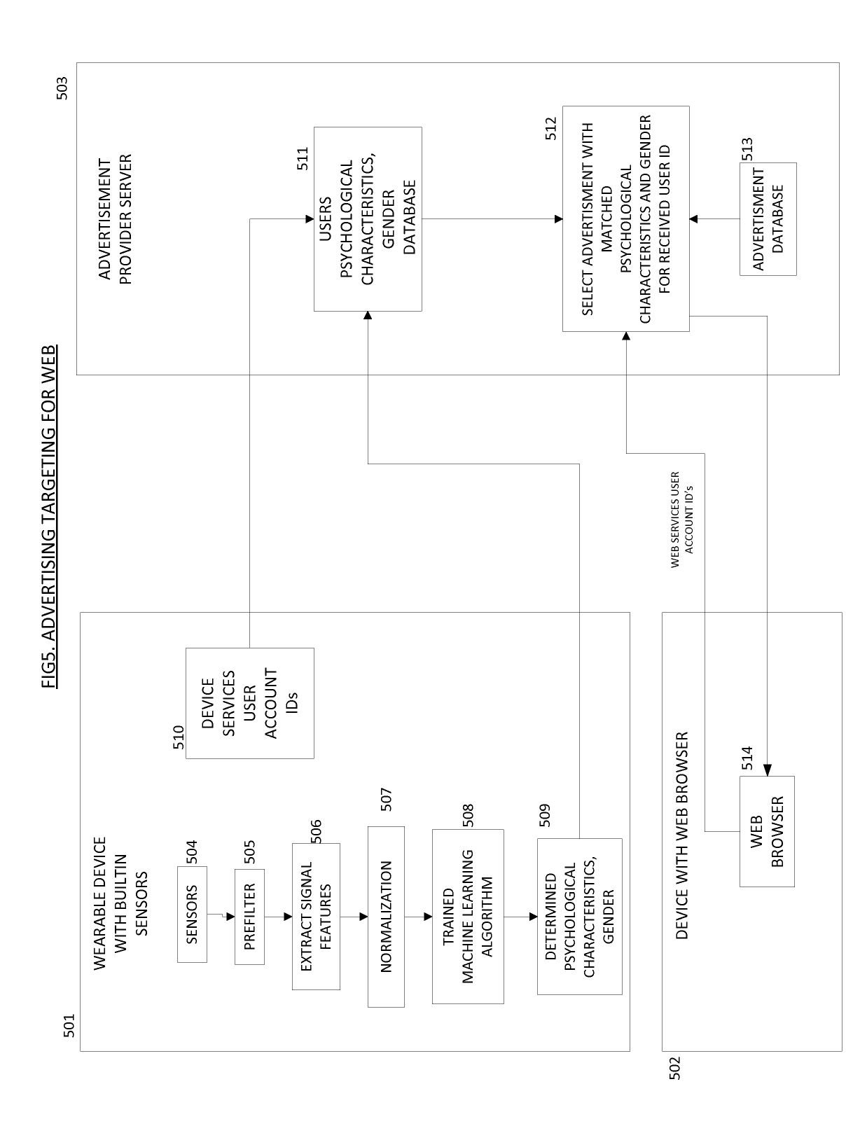

FIG. 5. Illustrates one of embodiments of invention in advertising targeting for Web.

Wearable device 501 with built-in sensors and display is used by an individual. The same device is used to access to third-party services which is also used by an individual to sign in to services within the browser on different devices 502 including desktop, TV with browsers, set-top boxes, tables, etc.

Motion sensors 504 register motion and pass data to filter 505. Filter 505 removes noises and useless data from the signal data. Then at the stage 506 prefiltered data is processed to extract signal features. The data as results of the processing at the stage 506 should be processed in the same manner as it does at stage 105 of FIG. 1.

Data from 506 is normalized at 507 if it is necessary for the chosen machine learning algorithm.

After normalization the data is passed as input to trained machine learning algorithm 508 to detect psychological characteristics and/or gender of the individual.

Determined psychological characteristics and/or gender is passed to advertisement provider server 503 in the bundle with account information 510 and stores in database 511.

At 512 the advertisement provider server 503 accepts request for the advertisement. Request should contain the web service account identifier. As request is received, the selecting advertisement with matched psychological characteristics and gender for received account identifier is performed. Selected advertisement sends to device 502 to display.

In one or more embodiments the stages 506,507,508,509 can be implemented at server side 502.

FIG. 5. Illustrates the method when advertisement preliminary is profiled with certain psychological or gender profile. In one or more embodiments the proposed technique of advertising targeting for Web can use statistical method in the same manner as illustrated in FIG. 4. to avoid the need of preliminary profiling of advertisement materials.

### Improving the Relevance of User Reviews

Many websites and programs on mobile devices (e.g. smartphones, tablets) provides functionality to collect user reviews of goods and services and display these reviews to other users.

Provided method is to store user reviews in the database also to save the psychological characteristics of the reviewer. Psychological characteristics of reviewers can obtained as described above, or traditional methods of determining the psychological characteristics. The goal of the review system is to provide the most relevant reviews to the users (viewers). The relevance can be increased by providing reviews when psychological characteristics of both the viewer and reviewer are similar.

For people with different psychological profiles does matter different aspects of the quality of the product and services. And their point of view is reflected in the reviews. For example, the relation between psychological characteristics and things, which person pays attention for restaurant may look like:
Extraverts: pay attention on the speed of service, how many people around.
Introverts: chamber or intimate atmosphere.
Logics: the order, prices, discount and special offers.
Ethics: politeness of the waiters, cordiality.
Intuits: waiter's advices when ordering.
Sensing: the taste of the dishes, quality of the food, specialties, comfort.
Irrational: the flexibility in making order, to combine different components of the dishes.
Rational: neatness and cleanliness, having the specialties.

The most difference between people which are extraverts and introverts. Currently, websites when showing reviews do not take in consideration individual characteristics of the reviewers and readers. But when psychological characteristics significantly differ the positive reviews do not find understanding to the reader, and negative reviews might point to aspects which is not important to the reader and usually do not impact to reader's choice. Showing non-relevant reviews unreasonably decreases interest to products and services.

By taking in consideration psychological characteristics of reviewers and readers is possible to increase relevancy the reviews which shown to certain reader. It helps to the reader to see the opinions which are close to his own opinion he would have about the product or services if he actually get it.

There are three methods of increasing relevancy of reviews provided in this invention:
1. Forming the rating based on weight coefficients (relevancy coefficients) that depends on the psychological characteristics of the reader and reviewer.
2. Sorting the reviews based on relevancy coefficients to show more relevant reviews first.
3. Filtering relevant reviews—showing only reviews with coefficient of relevancy more than given threshold.

1. When forming rating the relevancy coefficients are used. Formula of the rating with relevancy coefficients:

$$Rating = \frac{Σ ( {R}_{i} * {W}_{i} )}{Σ {W}_{i}}$$

where Ri—the i-reviewers rating, Wi—indicator of the relevancy the reviews of the reviewers to the reader, values from 0 to 1.

In simple case relevancy coefficients Wi can be determined on amount of equal psychological characteristics divided by the total amount of characteristics: Wi=C/N, where C is amount of equal psychological characteristics, N—the total amount of characteristics.

But different psychological characteristics impacts different to the relevancy. So by the practical found followings values of relevancy coefficients:

| Psychological characteristics of | Relevancy |
|---|---|
| Reviewer-Reader (using MBTI classification) | coefficients W |
| ENTP-ENTP, ISFJ-ISFJ, ESFJ-ESFJ, INTP-INTP, ENFJ-ENFJ, | 1 |
| ISTP-ISTP, ESTP-ESTP, INFJ-INFJ, ESFP-ESFP, INTJ-INTJ, | |
| ENTJ-ENTJ, ISFP-ISFP, ESTJ-ESTJ, INFj-INFj, ENFP-ENFP, ISTJ- | |
| ISTJ | |
| ENTP-ISFJ, ISFJ-ENTP, ESFJ-INTP, INTP-ESFJ, ENFJ-ISTP, | 0.8 |
| ISTP-ENFJ, ESTP-INFJ, INFJ-ESTP, ESFP-INTJ, INTJ-ESFP, | |
| ENTJ-ISFP, ISFP-ENTJ, ESTJ-INFj, INFj-ESTJ, ENFP-ISTJ, ISTJ- | |
| ENFP | |
| ENTP-INTP, ISFJ-ESFJ, ESFJ-ISFJ, INTP-ENTP, ENFJ-INFJ, | 0.9 |
| ISTP-ESTP, ESTP-ISTP, INFJ-ENFJ, ESFP-ISFP, INTJ-ENTJ, | |
| ENTJ-INTJ, ISFP-ESFP, ESTJ-ISTJ, INFj-ENFP, ENFP-INFj, ISTJ- | |
| ESTJ | |
| ENTP-ESFJ, ISFJ-INTP, ESFJ-ENTP, INTP-ISFJ, ENFJ-ESTP, | 0.7 |
| ISTP-INFJ, ESTP-ENFJ, INFJ-ISTP, ESFP-ENTJ, INTJ-ISFP, | |
| ENTJ-ESFP, ISFP-INTJ, ESTJ-ENFP, INFj-ISTJ, ENFP-ESTJ, | |
| ISTJ-INFj | |
| ENTP-ISTJ, ISFJ-ENFP, ESFJ-ISTP, INTP-ENFJ, ENFJ-INTP, | 0.8 |
| ISTP-ESFJ, ESTP-INTJ, INFJ-ESFP, ESFP-INFJ, INTJ-ESTP, | |
| ENTJ-INFj, ISFP-ESTJ, ESTJ-ISFP, INFj-ENTJ, ENFP-ISFJ, ISTJ- | |
| ENTP | |
| ENTP-ENFP, ISFJ-ISTJ, ESFJ-ENFJ, INTP-ISTP, ENFJ-ESFJ, | 1 |
| ISTP-INTP, ESTP-ESFP, INFJ-INTJ, ESFP-ESTP, INTJ-INFJ, | |
| ENTJ-ESTJ, ISFP-INFj, ESTJ-ENTJ, INFj-ISFP, ENFP-ENTP, ISTJ- | |
| ISFJ | |
| ENTP-ISFP, ISFJ-ENTJ, ESFJ-INTJ, INTP-ESFP, ENFJ-ISTJ, | 0.5 |
| ISTP-ENFP, ESTP-INFj, INFJ-ESTJ, ESFP-INTP, INTJ-ESFJ, | |
| ENTJ-ISFJ, ISFP-ENTP, ESTJ-INFJ, INFj-ESTP, ENFP-ISTP, ISTJ- | |
| ENFJ | |
| ENTP-ESFP, ISFJ-INTJ, ESFJ-ENTJ, INTP-ISFP, ENFJ-ESTJ, | 0.6 |
| ISTP-INFj, ESTP-ENFP, INFJ-ISTJ, ESFP-ENTP, INTJ-ISFJ, ENTJ- | |
| ESFJ, ISFP-INTP, ESTJ-ENFJ, INFj-ISTP, ENFP-ESTP, ISTJ-INFJ | |
| ENTP-ENTJ, ISFJ-ISFP, ESFJ-ESFP, INTP-INTJ, ENFJ-ENFP, | 0.6 |
| ISTP-ISTJ, ESTP-ESTJ, INFJ-INFj, ESFP-ESFJ, INTJ-INTP, ENTJ- | |
| ENTP, ISFP-ISFJ, ESTJ-ESTP, INFj-INFJ, ENFP-ENFJ, ISTJ-ISTP | |
| ENTP-INTJ, ISFJ-ESFP, ESFJ-ISFP, INTP-ENTJ, ENFJ-INFj, ISTP- | 0.3 |
| ESTJ, ESTP-ISTJ, INFJ-ENFP, ESFP-ISFJ, INTJ-ENTP, ENTJ- | |
| INTP, ISFP-ESFJ, ESTJ-ISTP, INFj-ENFJ, ENFP-INFJ, ISTJ-ESTP | |
| ENTP-INFJ, ISFJ-ESTP, ESFJ-INFj, INTP-ESTJ, ENFJ-ISFP, ISTP- | 0.6 |
| ENTJ, ESTP-ISFJ, INFJ-ENTP, ESFP-ISTJ, INTJ-ENFP, ENTJ- | |
| ISTP, ISFP-ENFJ, ESTJ-INTP, INFj-ESFJ, ENFP-INTJ, ISTJ-ESFP | |
| ENTP-ESTP, ISFJ-INFJ, ESFJ-ESTJ, INTP-INFj, ENFJ-ENTJ, | 0.9 |
| ISTP-ISFP, ESTP-ENTP, INFJ-ISFJ, ESFP-ENFP, INTJ-ISTJ, | |
| ENTJ-ENFJ, ISFP-ISTP, ESTJ-ESFJ, INFj-INTP, ENFP-ESFP, | |
| ISTJ-INTJ | |
| ENTP-ENFJ, ISFJ-ISTP, ESFJ-ENFP, INTP-ISTJ, ENFJ-ESFP, | 0.9 |
| ISTP-INTJ, ESTP-ESFJ, INFJ-INTP, ESFP-ESTJ, INTJ-INFj, ENTJ- | |
| ESTP, ISFP-INFJ, ESTJ-ENTP, INFj-ISFJ, ENFP-ENTJ, ISTJ-ISFP | |
| ENTP-ESTJ, ISFJ-INFj, ESFJ-ESTP, INTP-INFJ, ENFJ-ENTP, | 0.7 |
| ISTP-ISFJ, ESTP-ENTJ, INFJ-ISFP, ESFP-ENFJ, INTJ-ISTP, | |
| ENTJ-ENFP, ISFP-ISTJ, ESTJ-ESFP, INFj-INTJ, ENFP-ESFJ, | |
| ISTJ-INTP | |
| ENTP-ISTP, ISFJ-ENFJ, ESFJ-ISTJ, INTP-ENFP, ENFJ-INTJ, | 0.4 |
| ISTP-ESFP, ESTP-INTP, INFJ-ESFJ, ESFP-INFj, INTJ-ESTJ, | |
| ENTJ-INFJ, ISFP-ESTP, ESTJ-ISFJ, INFj-ENTP, ENFP-ISFP, ISTJ- | |
| ENTJ | |
| ENTP-INFj, ISFJ-ESTJ, ESFJ-INFJ, INTP-ESTP, ENFJ-ISFJ, ISTP- | 0.5 |
| ENTP, ESTP-ISFP, INFJ-ENTJ, ESFP-ISTP, INTJ-ENFJ, ENTJ- | |
| ISTJ, ISFP-ENFP, ESTJ-INTJ, INFj-ESFP, ENFP-INTP, ISTJ-ESFJ | |

2. When sorting reviews using relevancy coefficient, the reviews should be sorted for particular user to show the reviews with higher relevancy coefficient first. The relevancy coefficient determined using the method above.

What is claimed is:

1. A method for determining psychological characteristics or gender of a user comprising:
obtaining a data via motion sensors embedded in one or more wearable devices worn by the user;
determining the user's psychological characteristics or gender by processing the data using analytical approach.

2. The method of claim 1, wherein only one psychological characteristic is being determined.

3. The method of claim 1, wherein psychological characteristics and gender are being determined together.

4. A method for determining psychological characteristics or gender of a user comprising:
training a machine learning algorithm to classify motion sensor data into psychological characteristics or gender, wherein as training data is used a data related to a sample users set with psychological characteristics or gender determined by an expert or test and motion sensors data is obtained via motion sensors embedded in one or more wearable devices worn by the sample set users;
determining psychological characteristics of the user by applying the trained machine learning algorithm to data obtained via motion sensors embedded in wearable devices worn by the user.

5. The method of claim 4, wherein only one psychological characteristic is being determined.

6. The method of claim 4, wherein psychological characteristics and gender are being determined together.

7. A method of using psychological characteristics for advertisement targeting comprising:
displaying an advertisement to individual advertisement viewers having psychological characteristics matching to targeted psychological characteristics of the advertisement with defined threshold of accuracy.

8. The method of claim 7, comprising intentionally designing the advertisement to be targeted on people with certain psychological characteristics.

9. The method of claim 7, comprising identifying a psychological characteristics of the advertisement target audience further comprising: displaying the advertisement to viewers with known psychological characteristics; gathering statistics of the audience response for the advertisement; determine targeted psychological characteristics in accordance with response rate.

10. A method of improving relevance of user reviews comprising:
determining a relevancy coefficient depending on the psychological characteristics of a reader and a reviewer for every review;
displaying a relevancy coefficient next to every review.

11. The method of claim 10, wherein displaying of the relevancy coefficient is optional, and the reviews displaying ordered according to the value of relevancy coefficient;

12. The method of claim 10, wherein displaying of the relevancy coefficient is optional, and the reviews displaying filtered by value of relevancy coefficient more than defined threshold.

13. The method of claim 10, wherein displaying a rating calculated based on the relevancy coefficients and individual reviewers' ratings.
